(19) United States
(12) Patent Application Publication
(10) Pub. No.: US 2017/0000974 A1
(43) Pub. Date: Jan. 5, 2017

(54) Echogenic Indwelling Catheter

(71) Applicant: FERROSAN MEDICAL DEVICES SP. Z.O.O., Szczecin (PL)

(72) Inventors: Kai Lange, Birkerød (DK); Christian Rothe, Hundested (DK); Frederik Sonnenborg, Jyllinge (DK); Arnt Larsen Lund, Sorø (DK)

(21) Appl. No.: 15/038,937

(22) PCT Filed: Dec. 5, 2014
(86) PCT No.: PCT/DK2014/050417

§ 371 Date: May 24, 2016

Related U.S. Application Data
(60) Provisional application No. 61/912,262, filed on Dec. 5, 2013.

(51) Int. Cl. A61M 25/00; A61N 1/05; A61B 8/08; A61M 25/01; A61M 25/06

(52) U.S. Cl. CPC A61M 25/007; A61M 25/0017; A61M 25/0108; A61M 25/065; A61B 8/085; A61N 1/0504; A61M 2025/0186; A61M 2202/048

(57) ABSTRACT

The present application relates to an echogenic indwelling catheter, an assembly and a method for medicating a patient with medication such as anaesthetics or other suitable drugs. In one embodiment, the echogenic indwelling catheter comprises a catheter shaft (1) comprising a bore (2), a plurality of radial holes (3) distributed along at least a part of said catheter shaft and configured for delivery of medication, a guidewire (5b) attached to and/or within the bore of the catheter and extending beyond a distal end of the catheter shaft.

### FIELD OF INVENTION

The present disclosure relates to administration of fluid medication, in particular medication for management of pain, more specifically to an indwelling catheter for the administration of fluid medication. More specifically, the present disclosure relates to an echogenic indwelling catheter, an assembly and a method for medicating a patient with medication such as anaesthetics or other suitable drugs.

### BACKGROUND OF INVENTION

During a surgical operation, it may be desirable to insert a catheter in order to medicate a patient. For example, after surgery it may be required to provide a patient with a continuous and/or intermittent flow of medication through a catheter over a period of time where pain may occur.

A common procedure to medicate a patient is to introduce a hollow needle into the patient and insert a catheter through the needle. After insertion, the needle is retrogradely withdrawn, and the catheter stays inside the patient within proximity of nerves to which anaesthetic can be dosed from the catheter.

One problem with this approach is that the placement is very sensitive to movement or stretching of the patient, especially, if the catheter is an indwelling catheter that stays inside the patient for a long time, for example, more than 12 hours, or even several days. In other words, the success rate of placing a catheter near the desired nerves in a way that it is stays in place, especially for a long time, is very low. Another problem with the approach where the catheter is inserted through the needle is that the needle—when retracted—leaves space around the catheter. In some instances, a tunnel is formed, wherein leakage of drug along the catheter to the skin surface can occur, which is not desirable.

The placement of the catheter is very critical. If the catheter is displaced from its initial placement, the delivery is ineffective. As a consequence, the patient might suffer from extreme pain. In cases where the catheter is retrogradely displaced, the catheter cannot typically be pushed forward since it may be soft. Hence, the procedure of inserting a catheter might be required to be repeated and this takes additional time. Therefore, the existing treatment of postoperative pain using peripheral nerve blocks is inefficient compared to its potential.

If a peripheral nerve block fails, one quick solution to the problem is to drug the patient with pills. This is indeed not desirable due to systemic side effects. Another common, but only temporary, solution to the problem of placing the catheter is to push the needle and catheter a bit further in than the location of the nerves of interest. In this case, one can hope that the movement and stretching of the patient is such that the catheter is retrogradely displaced and ends in the proximity of the nerves. If the catheter is only retrogradely displaced a bit during needle removal, the catheter can actively be pulled in place, but if pulled too much, the problem recurs.

Some catheters have however an adjustable length, so the placement of the catheter can be adjusted a bit after displacement. These adjustable catheters are however very complex. Nevertheless, the adjustment, or rather re-adjustment, related to the insertion of the catheter is not the only problem.

Another problem is related to the insertion of the hollow needle and the catheter inside the patient. Since the catheter is to be placed within proximity of nerves, one has to identify the nerves, the needle and the catheter inside the patient.

One solution to this problem is to use ultrasound imaging and observe some echogenic parts of at least the needle and catheter. One problem with this approach is however that the possible echogenic features of at least the needle and the catheter are relatively small, and can therefore be difficult to identify. As a consequence, the identification and hence placement of the catheter inside the patient is unreliable. Furthermore, another problem is present in the retrograde withdrawal of the needle while using ultrasound imaging. One has to retract the needle, one has to compensate or maintain the position of the catheter while the needle is retracted, and one has to make sure that the actual position of the catheter is in the correct place using ultrasound imaging. In other words, the retrograde withdrawal is difficult to perform by a single person.

### SUMMARY OF INVENTION

The present disclosure addresses these problems by providing an echogenic indwelling catheter for administration of fluid medication, comprising a catheter shaft comprising a bore, a plurality of radial holes distributed along at least a part of said catheter shaft and configured for delivery of medication, and a guidewire attached to and/or within the bore of the catheter and extending beyond a distal end of the catheter shaft, wherein a distal end of the plurality of radial holes are positioned in front of a proximal end of the guidewire. Since the catheter is an echogenic catheter, it may comprise a part that is visible using ultra sound imaging. Furthermore, since the catheter is an echogenic indwelling catheter, it may be configured to be left temporarily or permanently inside a patient. The disclosed echogenic indwelling catheter is easy to identify and solves therefore the problem of identifying the catheter inside the patient using ultra sound imaging.

The present disclosure also relates to an assembly for administration of medication, comprising a hollow needle, wherein at least a part of it is echogenic, and the herein disclosed echogenic indwelling catheter, wherein the echogenic indwelling catheter has a cross sectional dimension that is identical or larger than the hollow needle. In this way, the space or channel formed in the tissue by the needle may be fully occupied by the catheter such that the problem of backflow and/or leaking medication from the injection area is solved. The needle may be a hollow needle and may be attached directly to the catheter having a proximal end, a distal end, and a guidewire.

In addition, the present disclosure provides a method for administration of medication to a target such as peripheral nerves, muscles blood vessels or fascia layers, comprising the steps of inserting a needle tip into an entrance area of a patient such that it forms an entrance perforation, aligning the needle tip in proximity of the target, pushing the needle further into the patient, such as into the tissue, such that the needle tip perforates and protrudes from an exit area of the patient such that it forms an exit perforation which is different from the entrance perforation in the patient, retracting the needle from the exit or entrance area of the patient while or after a catheter is guided into the patient from the exit perforation or the entrance perforation of the patient by the needle such that the catheter is positioned inside the patient and protruding both the entrance perforation and exit perforation, wherein the catheter comprises a shaft comprising a bore and a plurality of holes, injecting medication through the bore of the catheter and out of the plurality of holes in the catheter to the target.

The insertion of the catheter may be done by inserting a catheter through the exit perforation by attaching it to the needle tip, i.e. to the distal end, which is then pulled backwards out of the entrance perforation along with the catheter. Alternatively, the insertion of the catheter may be done by inserting a catheter through the entrance perforation by attaching it to the needle end, i.e. to the proximal end, which is then pushed forward out of the exit perforation. In any of these embodiments, the aligning or pushing step may comprise guidance by ultrasound imaging. In order to do so, it may be required that the needle or part thereof may be echogenic.

In addition, the present disclosure also relates to a method of inserting and positioning a catheter with integrated electrodes and/or sensors for electrical nerve stimulation, nerve modulation, recording of bio potentials and/or biometric measurements, since these functionalities also requires a precise and stable positioning in the tissue.

By having the herein disclosed catheter inside a patient such that it protrudes both the entrance perforation and the exit perforation, it may be possible to securely fixate the position of the catheter in both ends, such that it stays in place inside the patient and thereby solves the problem related to the placement of the catheter. Furthermore, since the catheter protrudes both the entrance perforation and the exit perforation, it may be easy to adjust or re-adjust the placement of the catheter (also after a prolonged time) by pulling its ends, such that the treatment of postoperative pain may be more efficient.

### DESCRIPTION OF DRAWINGS

FIG. 1.A shows a side view of an embodiment of an indwelling catheter of the present disclosure. The catheter shaft 1 comprises a bore 2 with fluid medication that flows out of a plurality of radial holes 3 such that a volume 4 remains filled with air during delivery of medication is formed within the bore. The volume 4 filled with air during delivery of medication is confined by the distance from the distal end of the plurality of holes 3 and the proximal end 5a of the guidewire 5b. The proximal end 5a of the guidewire 5b forms a blind end within the bore. In this indwelling catheter, the guidewire 5b is attached within the bore by welding or glue 6 on the catheter shaft. A cross sectional view is made from A-A and described in FIG. 1.B.

FIG. 1.B shows a cross-sectional view from A-A of an embodiment of an indwelling catheter of the present disclosure. Six radial holes 3 are distributed along the catheter shaft 1. The fluid medication in the bore 2 flows out of the radial holes 3.

FIG. 2 shows a side view of another embodiment of an indwelling catheter of the present disclosure. The catheter shaft 1 comprises a bore 2 with fluid medication that flows out of a plurality of radial holes 3. The bore comprises four obstructions 7 such that three volumes 4a of air are entrapped inside the bore. In this indwelling catheter, the guidewire 5b is attached within the bore by an obstruction 7 made by glue or similar curable substance inside the catheter shaft.

FIG. 3 shows a side view of yet another embodiment of an indwelling catheter of the present disclosure. The catheter shaft 1 comprises a bore 2 with fluid medication that flows out of a plurality of radial holes 3. The volume between the distal end of the plurality of holes 3 and the proximal end 5a of the guidewire 5b is filled with an echogenic material, which in this case is an echogenic coil 8.

FIG. 4 shows a side view of an embodiment of an assembly of the present disclosure, comprising an indwelling catheter and a hollow needle. The catheter shaft 1 comprises a bore 2 with fluid medication that flows out of a plurality of radial holes 3 such that a volume filled 4 with air during delivery of medication is formed within the bore. The volume filled 4 with air during delivery of medication is confined by the distance from the distal end of the plurality of holes 3 and the proximal end 5a of the guidewire 5b. The proximal end 5a of the guidewire 5b forms a blind end within the bore. In this indwelling catheter, the guidewire 5b is attached within the bore by glue 6 on the catheter shaft. The guidewire is inside the hollow and curved needle 9.

FIG. 5.A-FIG. 5.G illustrate an exemplary use of the presently disclosed catheter and assembly.

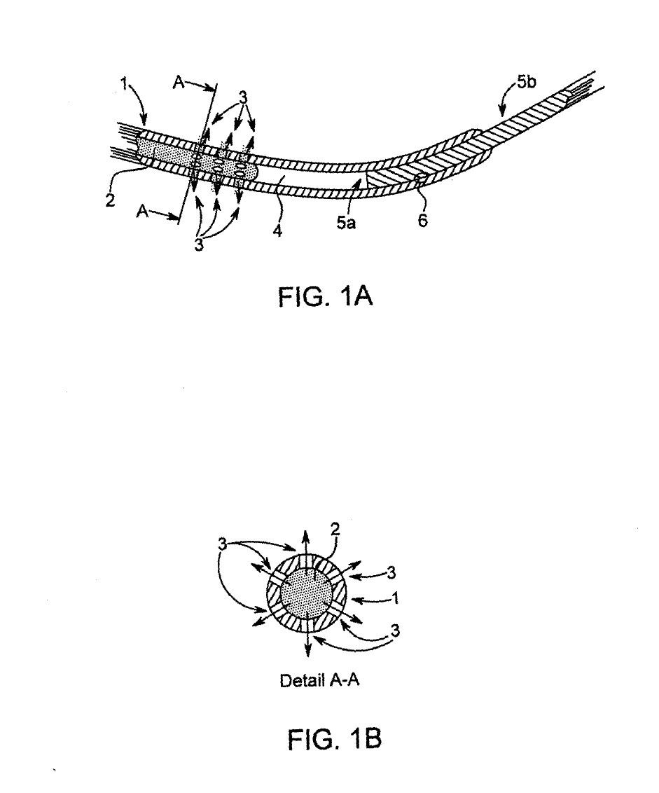

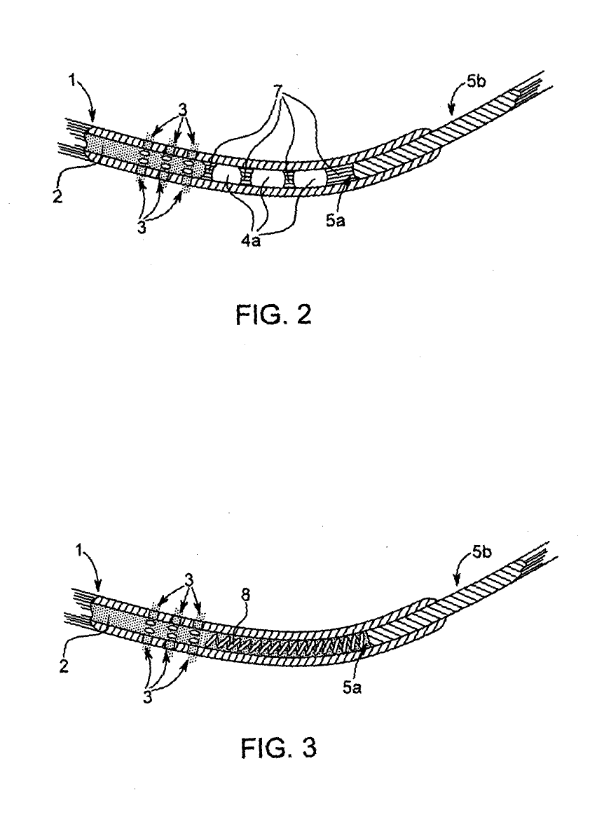

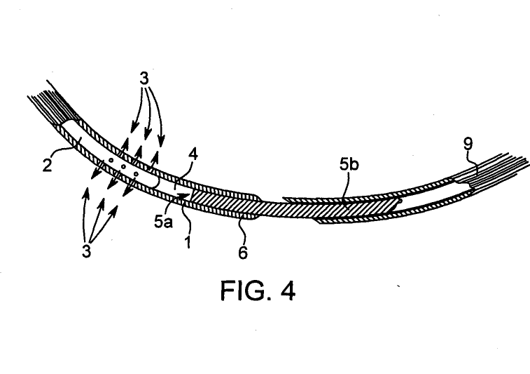

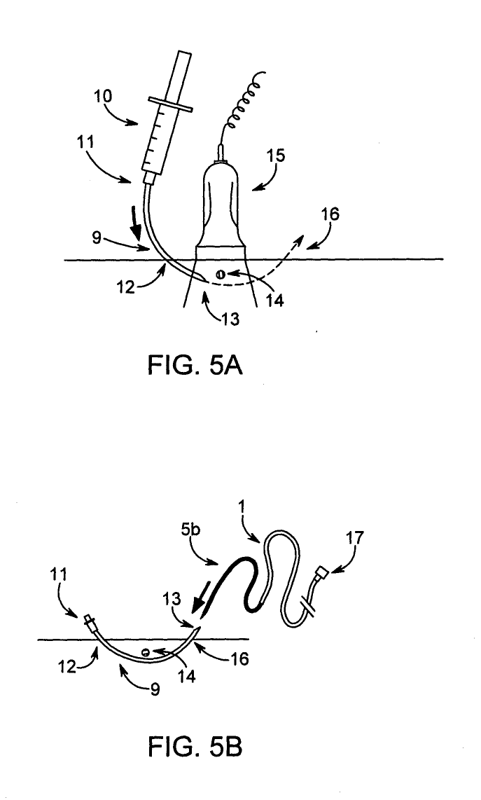

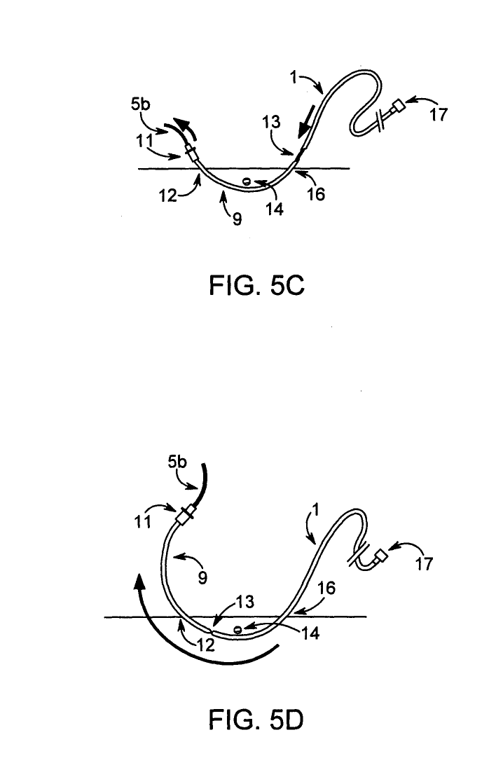

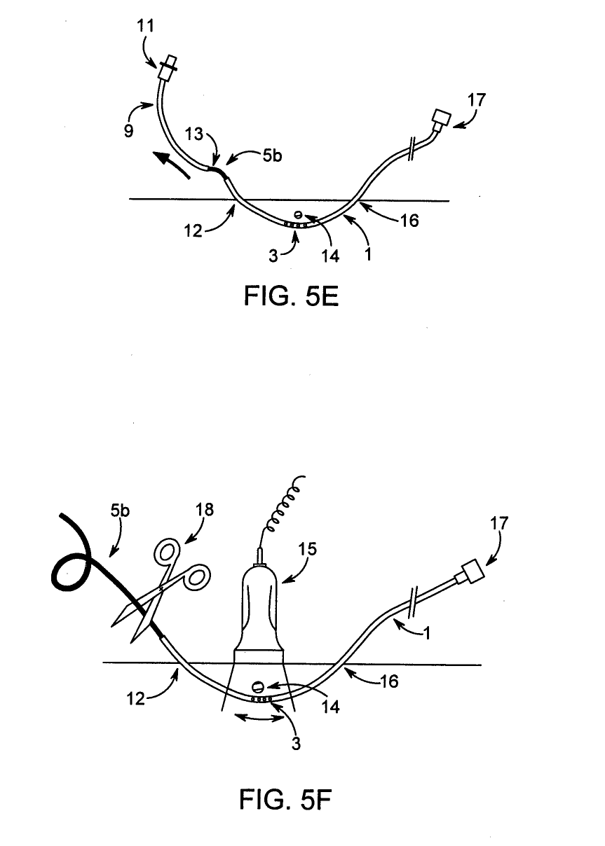

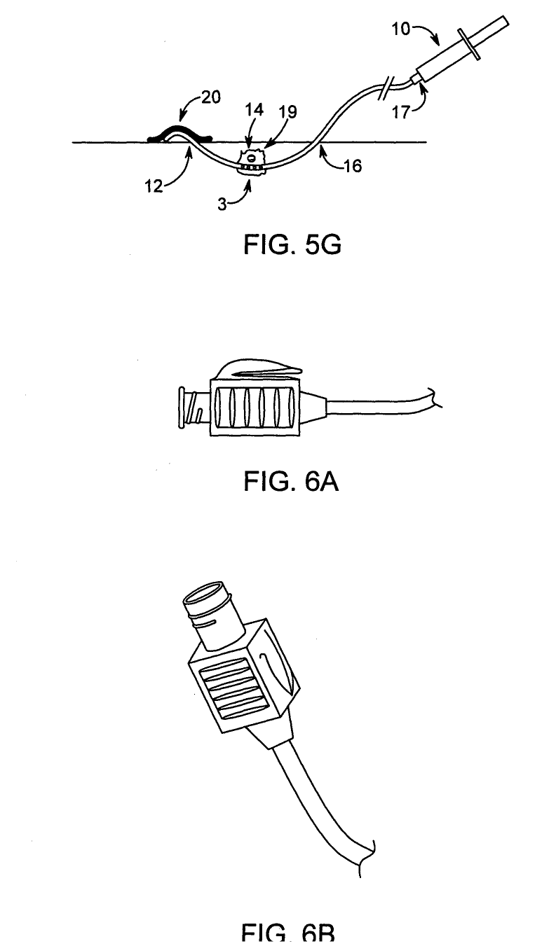

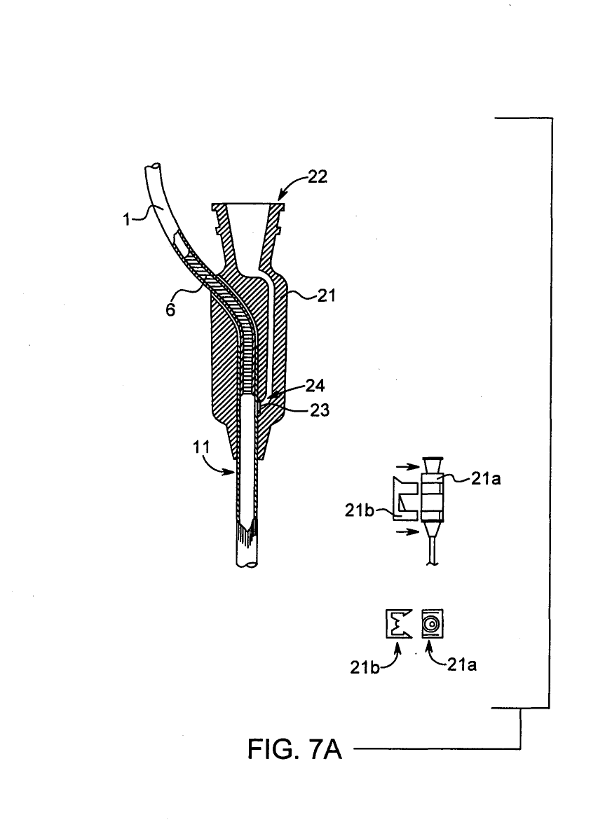

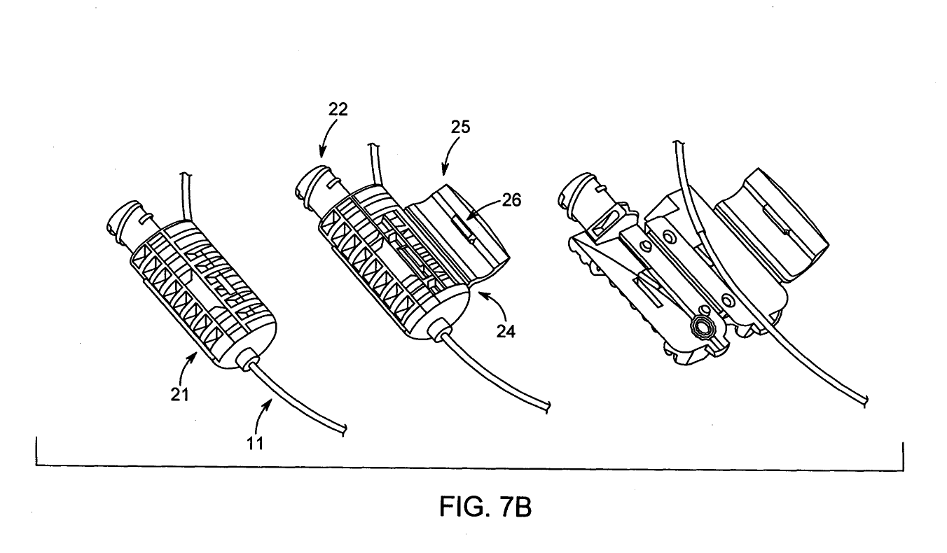

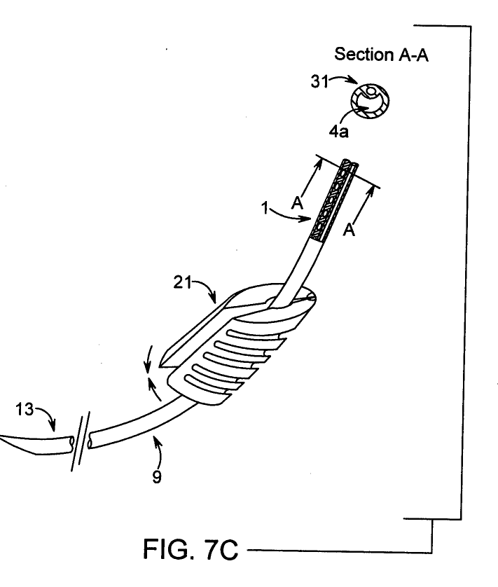

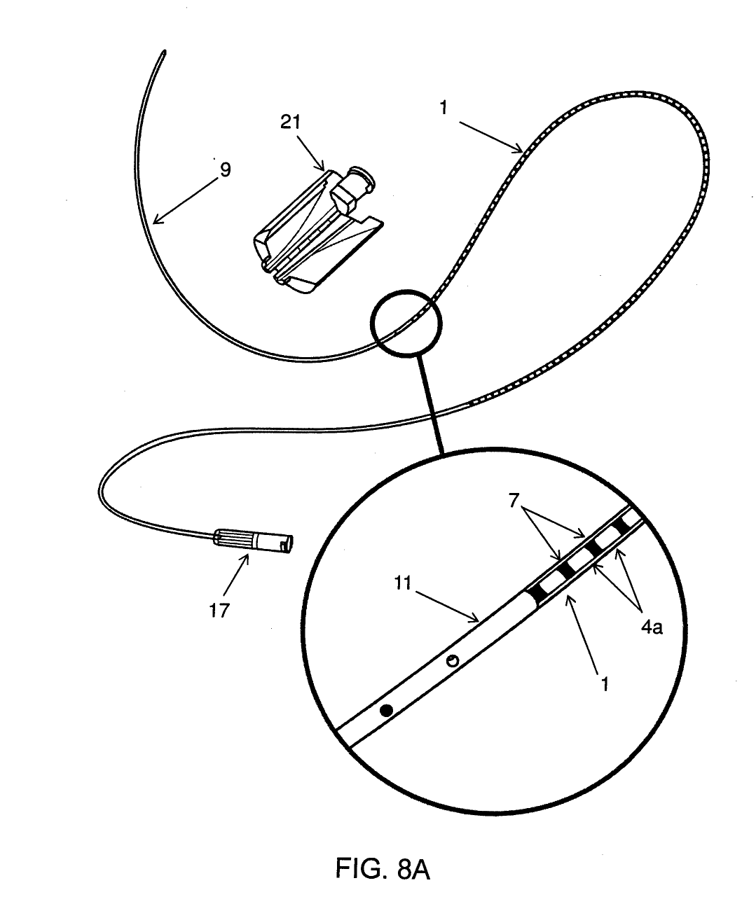

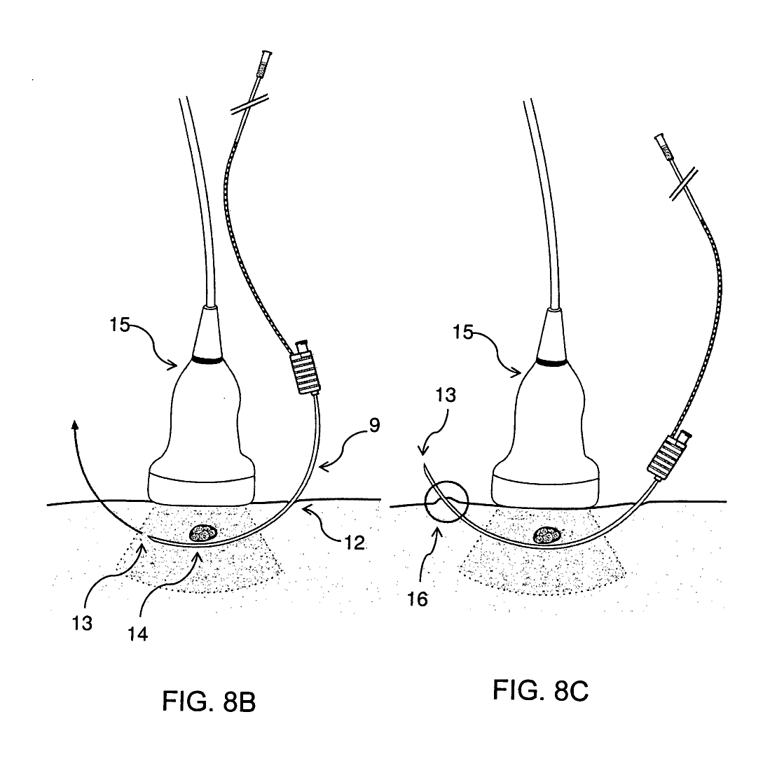

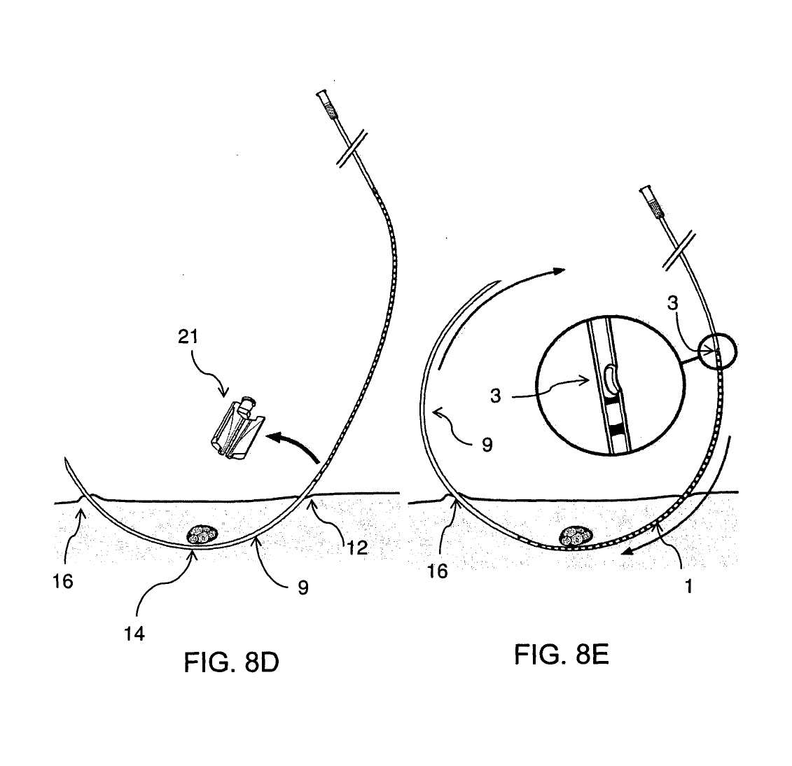

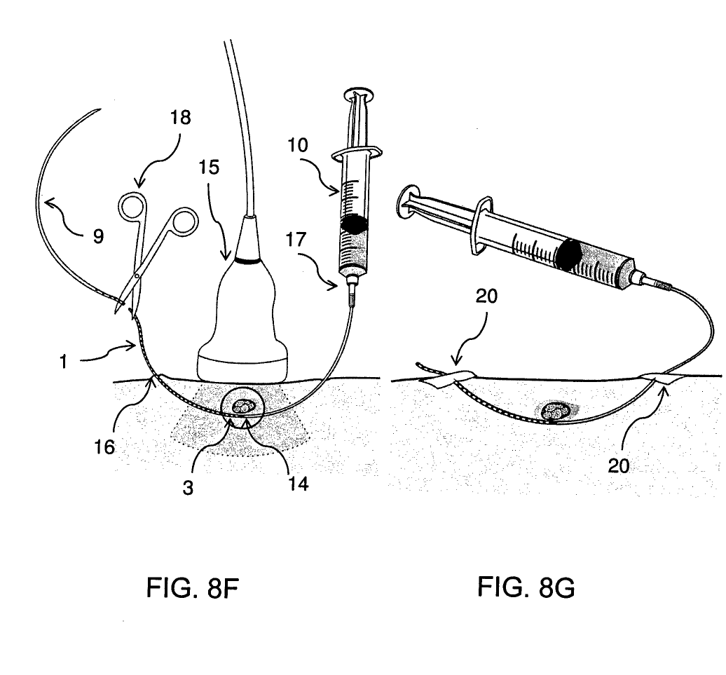

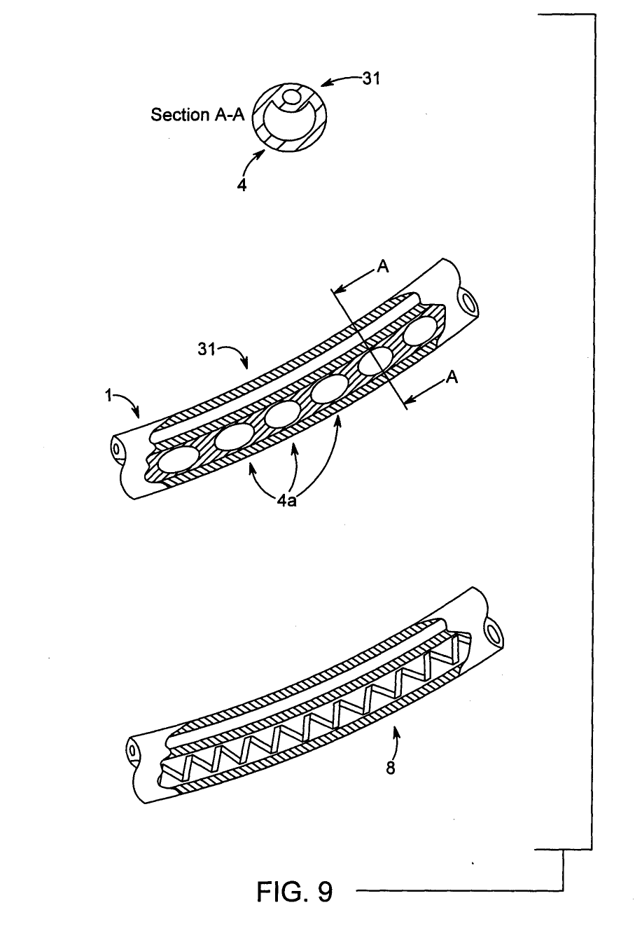

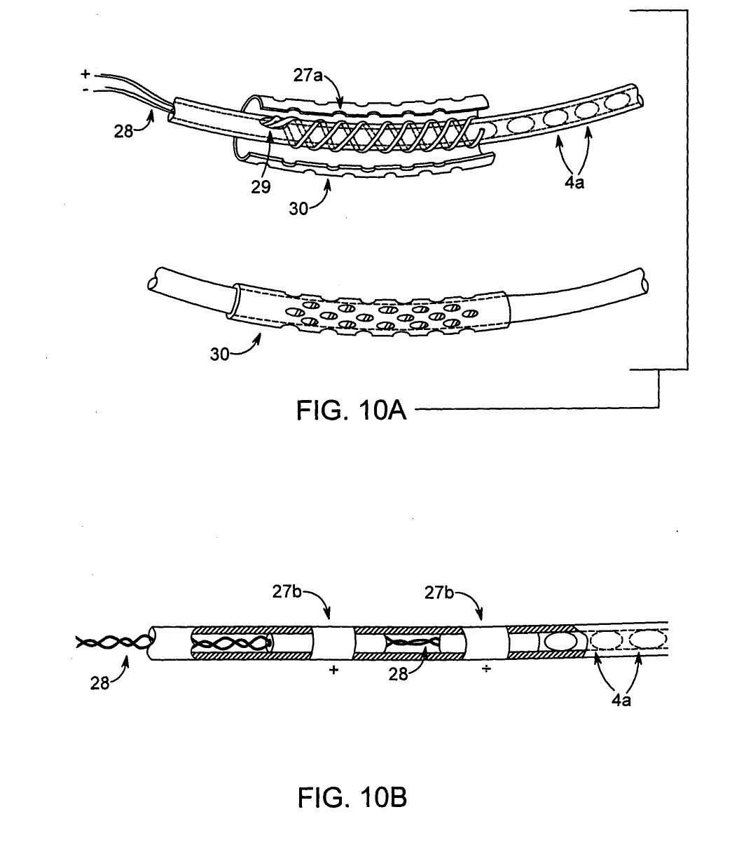

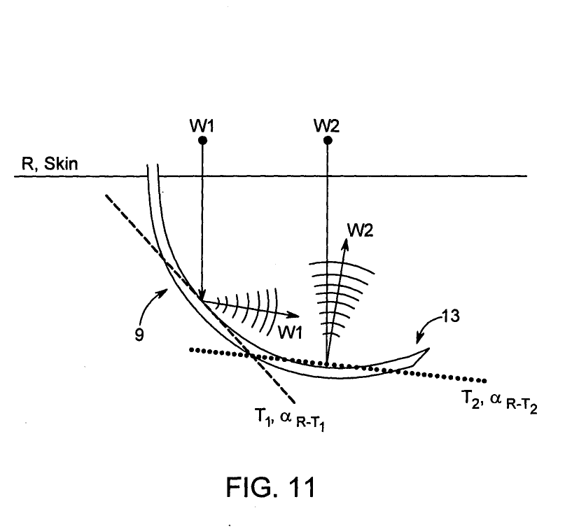

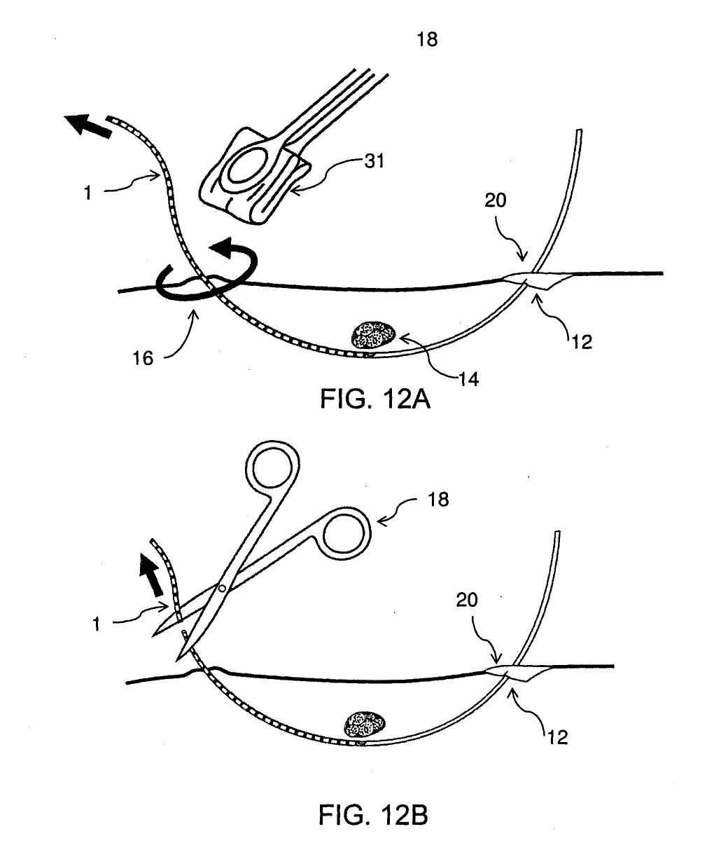

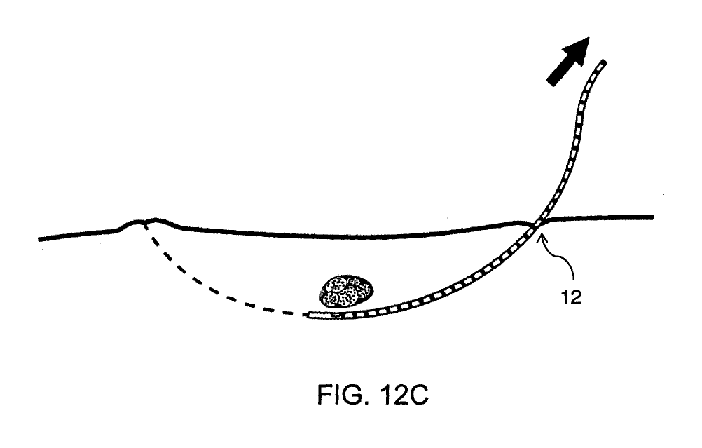

In FIG. 5.A, the syringe 10 is connected to the needle end 11 of the needle 9. The needle is curved and inserted into an entrance area 12 of a patient using the needle tip 13. The needle 9 is pushed into the patient and aligned in proximity of the nerve 14 using guidance of ultra sound imaging 15. The needle is pushed further into the patient such that the needle tip 13 perforates and protrudes an exit area 16 of the patient.

In FIG. 5.B, the needle 9 is inside the patient in proximity of the nerve 14 and protruding both the entrance area 12 and exit area 16 of the patient. The guidewire 5b, attached to the bore of the catheter, comprising a catheter shaft 1 and a proximal end 17, is guided into the needle tip 13. The guidewire may alternatively be guided into the needle end 11, but this approach is not shown.

In FIG. 5.C, the guidewire 5b, is guided further into the needle tip 13, through the needle 9 and out of the needle end 11.

In FIG. 5.D, the needle 9 is retracted from the patient from the side of the entrance area 12 such that the catheter shaft 1 is guided by the needle 9 and through the exit area 16 of the patient. The guidewire is advanced further through the needle until the catheter abuts the needle tip 13, such that the catheter follows the needle path while being retracted. The catheter is in this way placed in proximity of the nerve 14.

In FIG. 5.E, the needle 9 is completely retracted from the patient from the entrance area 12 such that the catheter shaft 1 is now inside the patient and protruding both the entrance area 12 and the exit area 16. The radial holes 3 of the catheter are to be placed in proximity of the nerve 14.

In FIG. 5.F, the guidewire 5b is shortened, in this case, with a scissor 18. The radial holes 3 of the catheter are placed in proximity of the nerve 14, guided by ultra sound imaging 15. The placement is achieved by alternating pulling the guidewire 5b and the proximal end of the catheter 17.

In FIG. 5.G, the catheter shaft 1 is inside the patient and protruding both the entrance area 12 and the exit area 16. The syringe 1 is connected to the proximal end of the catheter 17. The radial holes 3 of the catheter are in proximity of the nerve 14, such that fluid medication 19, injected from the syringe 1, flows out of the radial holes 3 and into the proximity of the nerve 14. A patch 20 is clamped to the catheter on the entrance area 12 such that the catheter is securely fastened.

FIG. 6.A shows a side view of an embodiment of the connector of the present disclosure.

FIG. 6.B shows a perspective of an embodiment of the connector of the present disclosure.

FIG. 7.A shows an embodiment of the connector of the present disclosure. The connector is positioned within a needle hub 21 as shown from the side. The catheter 1 as attached to the needle is blocked with glue or similar curable substance 6. Since it is important for the user to have the possibility for injection of medication such as local anaesthetics during insertion of the hollow needle, a by-pass channel connects a Luer Lock connector 22 on the hub to a hole 23 positioned on the side of the needle a distance further below the needle/catheter connection. Above the hole 23 is a sealing 24 between the hub 21 and the needle end 11. The needle hub 21 is detachable from the needle and catheter. To enable detachment of the needle hub 21, it is designed to consist of several parts, preferably two parts 21a and 21b, as shown from the side and above, pressed around the needle, fixating it in the hub and securing alignment of the hole in the side of the needle to the by-pass channel. The needle hub parts can be separated and removed.

FIG. 7B shows another embodiment of the Hub 21 with a Luer connector 22, consisting of only one part, and where the detachment from the needle end 11 is done by opening an incorporated hinge 24 and latch 25 with a snap lock 26. To open the hub, the snap is released and the latch is opened, successive allowing the hub to open so the needle can be removed from the hub.

FIG. 7C shows yet another embodiment of the hub 21 without internal by-pass channel and locking means. This embodiment acts as a needle gripper that clamps the needle 9 to facilitate handling and manipulation of the needle and needle tip 13. Instead of internal flow channels in the hub, the flow of medication is supplied through a second lumen 31 in the catheter 1. The additional lumen 31 runs in parallel to the echo genic markings 4a. When used, the hub is simply opened and by this released from the needle.

FIGS. 8.A-G illustrate embodiments and exemplary use of the presently disclosed catheter and needle assembly.

FIG. 8.A shows the complete assembly, where the distal end of the catheter 1 is attached directly to the needle end 11. The lumen of the distal end of the catheter is equipped with cavities filled with air 4a divided by a solid substance 7 as echogenic markers. The Hub 13 initially covers the mating zone (see FIG. 7.A & 7.B) during first part of procedure (here shown detached). At the proximal end of the catheter a Luer connector 17 enables fluid connection to auxiliary equipment e.g. a syringe or medication pump.

FIG. 8.B In this embodiment, the needle 9 is curved and inserted into an entrance area 12 of a patient using the needle tip 13. The needle 9 is pushed into the patient and aligned in proximity of the nerve 14 using guidance of ultra sound imaging 15.

FIG. 8.C The needle is pushed further into the patient such that the needle tip 13 perforates and protrudes an exit area 16 of the patient.

In FIG. 8.D the needle 9 is inside the patient in proximity of the nerve 14 and protruding both the entrance area 12 and exit area 16 of the patient. The hub 13 is detached from the needle and discarded.

FIG. 8.E The needle 9 is now pulled further all the way through the tissue and out of the exit hole 16, while the distal end of the catheter 1 is guided into the tissue in the trajectory of the needle. The attached catheter will be drawn behind the needle through the tissue until the distal end of the catheter is advanced beyond the exit site of the needle. As the catheter progresses into the tissue, the radial holes for dosage 3 of medication are pulled towards and into the tissue. The holes are positioned at the point where the echogenic marks most proximal to the Luer connector stops.

FIG. 8.F Under ultra sound guidance 15, the catheter 1 is advanced even further until the dosage holes 3 in the catheter can be positioned in proximity to the nerve 14 by manipulating the proximal and of the catheter with the Luer connector 17 or the distal end of the catheter emerging from the skin at the exit site 16. A syringe is connected to the Luer connector 17 to perform hydro dissection or confirm dosage hole position by injection of anaesthetics or saline. When the optimal position of the catheter is reached, a bolus can be injected proximal to the nerve. The catheter is then cut a suitable distance from the skin with a sterile pair of scissors 18. The needle 9 is discarded.

FIG. 8.G By securing with adhesive patches 20, the catheter at both the entrance- and exit area, the position of the dosage holes are kept in a stable position proximal to the nerve. Future injections can be performed without further preparations. If the position of the catheter is disturbed, the patches can be removed and the catheter repositioned by repeating step 8F.

FIG. 9 Illustrates an example of the catheter according to the present invention with an additional lumen 31 with an air filled volume 4, enclosed cavities 4a or similar echogenic marks such as a coil 8 running in parallel with a lumen used for support/convey the primary functionality of the catheter, e.g. flow of medication, conveying optical/electrical cords and/or means for mechanical actuation equipment in or auxiliary of the catheter.

FIGS. 10.A-B Illustrate examples of catheters according to the present invention with integrated electrodes 27 for nerve stimulation, nerve modulation, recording of bio potentials and/or biometric measurements.

FIG. 10.A Shows one example, where the electrodes 27a consists of electric conducting wires 28 that runs inside the catheter lumen and protrudes to the outer surface via an opening 29 in the catheter at the proximal end of the echogenic markings 4a. The wires can be insulated or partly insulated. From the point where they emerge to the outer surface, the wires are able to conduct a current or bio potential to or from their surroundings. To enhance the exposed surface of the electrodes, they may be arranged in a way other than linear e.g. as a helical patterns as shown. There may be two or more polarities. This example illustrates two polarities. The helical coil may be protected with a sheath of e.g. shrink wrap/tube 30.

FIG. 10.B shows another example where the electrodes 27b consist of specific zones of the catheter with a conductive surface. In this example the zones are made of integrated tubular metal parts with the catheter glued to each ends, positioned at the proximal end of the echogenic markings 4a. The metal parts can be hollow to allow internal wires going through them and the catheter lumen. Instead of integrated metal parts, the conductive zones may consist of conductive ink printed on the outer surface of the catheter and connected to internal wires or other means of electrical connections.

FIG. 11 Illustrates the method according to the present invention, where increased/reduced angle is shown between ultra sonic probe window and the inserted needle. When an ultra sonic sound wave W1 & W2 are emitted from the transducer from the skin surface R, the waves travels into the tissue. When hitting structures, the waves are reflected in more or less diffuse angles. The needle 9 has a well defined surface, which also reflects the waves. A simplified model of the situation is seen in FIG. 12. It shows two scenarios. A wave W1 is emitted and hits the needle at a point where the tangent to the curve T1 have an angle α_{R-T1} to the skin surface R. The angle of reflection is too steep, meaning that the sound waves W1′ will not be reflected back to the transducer head. Another wave W2 hits the needle at a point near the tip 13. Here the tangent T2 has an angle α_{R-T2} that is more parallel compared to the skin surface R, meaning that more of the waves W2 (energy) is reflected back into the ultra sonic transducer and hence the needle is visible on the screen. As a rule of thumb, the needle will not be visible if the angle of its axis (straight needle) or tangent in a specific point (curved) compared to the transducer head is above 45°.

FIG. 12 Illustrates the method according to the present invention, where a removal of the catheter from the patient is shown.

FIG. 12.A Illustrating that while the patch 20 at the insertion hole 12 is kept in place, the fixation patch at the exit hole 16 is removed. The distal end of the catheter 1 is gently pulled to retract it 5-10 mm from the skin at the exit hole 16. The exposed catheter and the surrounding skin are cleaned with a swap soaked with disinfectant agent 31.

FIG. 12.B Still keeping the distal end of the catheter 1 stretched, it is cut with a pair of scissors 18 just above skin level. The fixation patch 20 at the insertion hole 16 is removed.

FIG. 12.C Illustrating the proximal part of the catheter being retracted through the insertion hole 12

### DETAILED DESCRIPTION OF THE INVENTION

As stated previously, the reason for the echogenic indwelling catheter to have a cross sectional dimension that is identical or larger than the hollow needle is that the catheter may fill up the space made by the needle to avoid backflow and/or leaking of medication. It may therefore not be possible to insert the catheter into the hollow needle.

One purpose of the present invention is to provide the echogenic indwelling catheter suitable for adjusting, re-adjusting and verification of the placement of the echogenic indwelling catheter in a patient. This is for improving the success rate of placing a catheter in a patient, but also to make the treatment of postoperative pain more efficient.

In one embodiment of the disclosure, the medication is anaesthetic or any suitable drug, preferably a local anesthetic drug. The anaesthetic may for example be chosen from the group of ropivacaine, bupivacaine, mepivacaine, lidocaine and/or any other local anesthetic drug.

In one embodiment of the disclosure, a proximal end of the catheter is configured to be connected to an auxiliary device such a syringe or a pump in order to inject medication.

The distal end of the catheter is preferably configured for passing through human or animal tissue.

In one embodiment of the disclosure, the area of the catheter proximal to and/or distal to the plurality of radial holes comprises echogenic marks such as echogenic coatings, echogenic coils, echogenic cavities, echogenic solids or echogenic indents or similar shifts in geometry/diameter, causing varying reflection of the ultra sound waves, such that they can be observed using ultra sound imaging due to the shifts in the reflected energy and derived by this, the contrast of the generated picture. Hence, the position of the catheter can be determined more precisely in proximity of the anatomical target such as peripheral nerves, muscles, blood vessels or fascial layers.

In one embodiment of the disclosure, the catheter shaft is made of a generally flexible polymeric composition such as polyurethane, nylon, polyethylene, PTFE or silicone.

The length of the catheter may be up to 100 mm, 200 mm, 300 mm, 400 mm, 500 mm, 600 mm, 700 mm, 800 mm, 900 mm or up to 1000 mm. The length of the catheter may be 100 mm, 200 mm, 300 mm, 400 mm, 500 mm, 600 mm, 700 mm, 800 mm, 900 mm or 1000 mm. The length of the catheter may be less than 100 mm, 200 mm, 300 mm, 400 mm, 500 mm, 600 mm, 700 mm, 800 mm, 900 mm or less than 1000 mm. The required length of the catheter may depend on the size or part of the patient.

In one embodiment of the disclosure, the catheter shaft comprises a weakened zone such that the catheter is configured to divide into two blunt pieces if accidentally subjected to excessive load or intentionally prior to removal of the catheter to prevent pulling the distal part of the catheter all the way back through the tissue. The weakening of the catheter leading to dividing can be active, e.g. caused by mechanical, electro mechanical or magnetic means or passive, e g. if the weakening is caused by degrading of a bio-absorbable substance or simply a zone of the catheter with less pull strength. If divided, it may be such that the two blunt pieces are configured such that they cannot cut the patient.

In one embodiment of the disclosure, the guidewire is attached to and/or within the bore by means of welding or glue.

In one embodiment of the disclosure, the guidewire is a stiff thread. The diameter of the guidewire may be 0.1 mm, 0.2 mm, 0.3 mm, 0.4 mm, 0.5 mm or 0.6 mm. The diameter of the guidewire may be shorter than 0.1 mm, 0.2 mm, 0.3 mm, 0.4 mm, 0.5 mm or shorter than 0.6 mm. The diameter of the guidewire may be greater than 0.1 mm, 0.2 mm, 0.3 mm, 0.4 mm, 0.5 mm or greater than 0.6 mm. The length of the guidewire may be 100 mm, 200 mm, 300 mm, 400 mm or 500 mm. The length of the guidewire may be shorter than 100 mm, 200 mm, 300 mm, 400 mm or shorter than 500 mm. The length of the guidewire may be greater than 100 mm, 200 mm, 300 mm, 400 mm or greater than 500 mm. The required length of the guidewire may depend on the size or part of the patient.

In one embodiment of the disclosure, the guidewire is made of an echogenic material such that it can be observed using ultrasound imaging. Furthermore, the guidewire may be at least partly flexible, such that it can be guided inside a patient. Also, the guidewire may be partly solid, such that it can be guided inside a patient. Even further, the guidewire may be an extending part of the same body of said catheter shaft such that the guidewire guides the catheter.

In one embodiment of the disclosure, the echogenic indwelling catheter is configured such that a volume between the distal end of the plurality of radial holes and the proximal end of the guidewire forms a blind end such that the volume remains filled with air during delivery of medication. Since air is strongly echogenic, the volume of air may be observed using ultra sound imaging. The distance between the distal end of the plurality of radial holes and the proximal end of the guidewire may be longer than 5 mm. The radial holes may have a diameter of 0.2 mm-0.5 mm or 0.1 mm to 0.6 mm. In one embodiment of the disclosure, the bore of the catheter comprises one or more obstructions distal to the plurality of holes such that one or more volumes of air or other echogenic materials are entrapped inside the bore. The distance along the bore within the volume(s) may be greater than 5 mm. The distance of along the bore within the obstructions may be 3 mm-5 mm or 1 mm-7 mm or greater than 7 mm. The obstructions may be formed by welding or UV-cured glue.

In one embodiment of the disclosure, the volume is partly or completely filled with a metal coil, foil or any other echogenic material. This volume may be the volume filled with air during delivery of medication or the volume that is permanently entrapped inside the bore.

In another embodiment of the disclosure, the echogenic indwelling catheter is configured to include two or more parallel lumens, such that one lumen contain said echogenic means as described above, while one or more parallel lumens convey flow of one or more types of medication e.g. a primary and secondary anaesthetic, and/or optical/electrical cords and/or means for mechanical actuation equipment in or auxiliary to the catheter. Said additional lumen could also facilitate insertion of the catheter by means of a stylet.

In one embodiment of the disclosure, the position of the plurality of radial holes is configured to avoid filling up said volume by medication. This may be achieved by having the radial holes in a distance in front of the blind end, such that the air cannot be displaced by the fluidic medication as long as the diameter of the bore of the catheter is configured to have a capillary effect.

As stated previously, the echogenic indwelling catheter may be used in an assembly with a hollow needle. The hollow needle may have a diameter of 0.9 mm-1.2 mm. In one embodiment of the disclosure, the hollow needle is straight. The needle may be 80 mm or 160 mm, longer than 160 mm or shorter than 160 mm. The needle may be useful for insertion in a leg or an arm or any part of a human or animal, through the skin or intraoperatively.

In another embodiment of the disclosure, the hollow needle is curved. One advantage of a curved needle is that it can be used to go through a patient in an entrance area and out on an exit area close to the entrance area, for example in a shoulder. Another advantage of said curvature is the improved reflection of ultra sonic waves, caused by the successively reduced angle between the needle and the contact surface/window of the ultrasonic probe. Said angle is reduced along the slope of the curved needle, meaning that the needle is almost parallel to the probe window at the tip of the needle. The more parallel, i.e. the smaller the angle, the better reflection is achieved. Thus, improved visualization of the needle, especially the needle tip is achieved. Using traditional straight needles, the reflection angle is constant along the length of the needle shaft, hence making the visibility of the needle strongly dependent of an initially correct insertion angle compared to the probe. Using a curved needle, the probability of getting a strong reflection at some point on the slope near the tip is very high, even though the needle proximal to the insertion point is steep, causing poor reflection and hence poor visualization. The radius of curvature may be 50 mm to 120 mm, smaller than 50 mm or greater than 120 mm. Such a needle variation may be of particular relevance for blocking nerves, such as Scalene-, Saphenous- or Tibial nerves.

In one embodiment, the needle tip is grinded to improve skin perforation. The angle of the grind may be 20 degrees, less than 20 or more than 20. The grinded surface can face inwards or outwards, the latter to improve skin perforation from the inside out. Due to the curved trajectory of the needle the tip will appear pointier towards the skin when the grinded surface faces outwards compared to the centre of curvature.

It is not always easy to identify nerves inside a patient, and therefore it might be advantageous to be able to stimulate the nerves. Hence, in one embodiment of the disclosure, the hollow needle is configured for providing electrical stimulation to peripheral nerves.

In one embodiment of the disclosure, the hollow needle comprises a needle shaft, a proximal end, a distal end, a non-conductive layer along a first part of said needle shaft, a conductive layer along a second part of said needle shaft and a needle tip.

In one embodiment of the disclosure, the assembly further comprises a syringe comprising medication and a pair of patches, so that medication can be injection into the patient and so that the catheter can attached to the patient.

The proximal end of said hollow needle preferably comprises a connector such as a Luer Lock fitting. The connector has fastening and locking means of the catheter such as a clamp, a slot, or a plug.

In another embodiment, said connector is a hub that encapsulates the connecting area between said hollow needle and catheter. The catheter may be directly or indirectly attached to the needle. The hub may be configured with a handle/gripping surfaces to improve manual handling of needle and catheter and said hub is detachable from the needle and catheter assembly to facilitate that the assembly can be pulled through the tissue as a unit.

In one embodiment of the disclosure, the aligning, pushing or retracting step comprises guidance of electrical nerve stimulation. This may be in order to identify the nerves of interest.

In one embodiment of the disclosure, the aligning, pushing or retracting comprises injecting the medication through the needle and out of the needle tip. This may be in order to medicate the patient in order to manage pain during the aligning or the pushing of the needle.

In one embodiment of the disclosure, the retracting step comprises removing the syringe from the needle end so that the needle end is able to receive another device such or part thereof, for example a guidewire.

In one embodiment of the disclosure, the retracting step comprises leading a guidewire of the catheter into said needle tip or needle end, through said needle and out of the needle end or needle tip.

In one embodiment of the disclosure, the retracting step comprises pulling the guidewire of the catheter from the needle end or needle tip.

In one embodiment of the disclosure, the injection step comprises connecting said syringe to said proximal end of said catheter. During injection, at least a part of the catheter may be in the proximity of the target by inspecting an echogenic part of the catheter by ultrasound imaging. In one embodiment of the disclosure, the part of said catheter comprises a volume between the distal end of the plurality of holes and the proximal end of the guidewire, wherein the volume remains filled with air during injection of medication.

In one embodiment of the disclosure, the injection step comprises aligning plurality of holes of the catheter in the proximity of the target by inspecting a plurality of echogenic marks around the plurality of holes by ultrasound imaging.

In one embodiment of the disclosure, the injection step comprises adjusting the catheter by pulling the guidewire of the catheter or the proximal end of the catheter.

Instead of having a traditional catheter that cannot be pushed further into the patient or even a catheter with an adjustable length, one may simply pull the proximal end of the catheter into the correct position. In case, where the catheter is pulled too much in one direction, one may now pull the catheter in the other direction and into the correct position. If in case, the catheter displaces over time, one may now re-adjust the position of the catheter by alternating between pulling the guidewire of the catheter and pulling the proximal end of the catheter until satisfied. This can be done with one hand while holding an ultrasound imaging device with the other hand.

In one embodiment of the disclosure, the injection step comprises fixing the guidewire to the needle end or needle tip such that the guidewire and hence the catheter follows the needle.

In another embodiment, the catheter is permanently attached direct to the needle. The needle is pulled all the way through the tissue through an insertion hole and an exit hole and hence the catheter is pulled completely through the tissue as well. After re-emerging of the catheter through the exit hole, the needle is separated from the catheter, leaving only the catheter in the tissue.

In one embodiment of the disclosure, the injection step comprises attaching a pair of patches to the catheter on the entrance area and the exit area such that the catheter is adjustable.

In one embodiment of the disclosure, the injection step comprises observing holes of the catheter in the proximity of the target by inspecting a plurality of echogenic marks of the catheter by ultrasound imaging.

In one embodiment of the disclosure, the injection step comprises clamping the patches to the catheter on the entrance area and the exit area such that said catheter is securely fastened.

In one embodiment of the disclosure, the injection step comprises releasing patches from the catheter on the entrance area and the exit area such that the catheter is adjustable.

1. An echogenic indwelling catheter for administration of fluid medication, comprising:
a catheter shaft comprising a bore;
a plurality of radial holes distributed along at least a part of said catheter shaft and configured for delivery of medication,
wherein said bore of the catheter comprises obstructions, each obstruction comprising a solid substance distal to the plurality of holes such that one or more volume(s) of air or another echogenic material is/are permanently entrapped between the obstructions and inside the bore; and
a guidewire attached to and/or within the bore of said catheter and extending beyond a distal end of said catheter shaft, wherein a distal end of said plurality of radial holes are positioned in front of a proximal end of said guidewire.

2. (canceled)

3. The echogenic indwelling catheter according to claim 1, wherein said medication is anaesthetic or any suitable drug.

4-7. (canceled)

8. The echogenic indwelling catheter according to claim 1, wherein said catheter shaft comprises a weakened zone.

9-10. (canceled)

11. The echogenic indwelling catheter according to claim 1, wherein said guidewire is made of an echogenic material.

12-17. (canceled)

18. An assembly for administration of medication, comprising:
a hollow needle, wherein at least a part of it is echogenic, and
an echogenic indwelling catheter for administration of fluid medication, comprising:
i. a catheter shaft comprising a bore;
ii. a plurality of radial holes distributed along at least a part of said catheter shaft and configured for delivery of medication, wherein said bore of the catheter comprises obstructions, each obstruction comprising a solid substance distal to the plurality of holes such that one or more volume(s) of air or another echogenic material is/are permanently entrapped between the obstructions and inside the bore; and
iii. a guidewire attached to and/or within the bore of said catheter and extending beyond a distal end of said catheter shaft, wherein a distal end of said plurality of radial holes are positioned in front of a proximal end of said guidewire,
wherein said indwelling echogenic catheter has a cross sectional dimension that is identical or larger than the hollow needle.

19. (canceled)

20. The assembly according to claim 18, wherein said hollow needle is straight.

21. The assembly according to claim 18, wherein said hollow needle is curved.

22. The assembly according to claim 18, wherein said hollow needle is configured for providing electrical stimulation to peripheral nerves.

23. The assembly according to claim 18, wherein said hollow needle comprises:
a needle shaft,
a proximal end,
a distal end,
a non-conductive layer along a first part of said needle shaft,
a conductive layer along a second part of said needle shaft, and
a needle tip.

24. The assembly according to claim 18, further comprising:
a syringe comprising medication, and
a pair of patches.

25-27. (canceled)

28. A method for administration of medication to a target, comprising the steps of:
inserting a needle tip into an entrance area of a patient such that it forms an entrance perforation;
aligning said needle tip in proximity of the target;
pushing said needle further into said patient such that said needle tip perforates and protrudes from an exit area of said patient such that it forms an exit perforation which is different from said entrance perforation in said patient;
retracting said needle from said exit area or entrance area of said patient while or after a catheter is guided into said patient from said exit perforation or entrance perforation of said patient by said needle such that said catheter is positioned inside said patient and protruding both said entrance perforation and exit perforation, wherein said catheter comprises:
i. a shaft comprising a bore, and
ii. a plurality of holes; and
injecting medication through said bore of said catheter and out of said plurality of holes in said catheter to said target.

29. The method according to claim 28, wherein said target is peripheral nerves, muscles, blood vessels or fascial layers.

30-33. (canceled)

34. The method according to claim 28, wherein said catheter is an echogenic indwelling catheter according to any of the preceding claims.

35-40. (canceled)

41. The method according to claim 28, wherein said retracting step comprises leading a guidewire of said catheter into said needle tip or needle end, through said needle and out of said needle end or needle tip.

42. The method according to claim 28, wherein said retracting step comprises pulling said guidewire of said catheter from said needle end or needle tip.

43. (canceled)

44. The method according to claim 28, wherein said injection step comprises aligning at least a part of said catheter in the proximity of said target by inspecting an echogenic part of said catheter by ultrasound imaging.

45-47. (canceled)

48. The method according to claim 28, wherein said injection step comprises adjusting said catheter by pulling said guidewire of said catheter or said proximal end of said catheter.

49. (canceled)

50. The method according to claim 28, wherein said injection step comprises attaching a pair of patches to said catheter on said entrance area and said exit area such that said catheter is adjustable or re-adjustable.

51. (canceled)

52. The method according to claim 28, wherein said injection step comprises clamping said patches to said catheter on said entrance area and said exit area such that said catheter is securely fastened.

53. The method according to claim 28, wherein said injection step comprises releasing patches from said catheter on said entrance area and said exit area such that said catheter is adjustable or re-adjustable.

54. (canceled)
